(19) United States
(12) Patent Application Publication
(10) Pub. No.: US 2016/0338949 A1
(43) Pub. Date: Nov. 24, 2016

(54) STABILIZED GASTRORETENTIVE TABLETS OF PREGABALIN

(71) Applicant: SUN PHARMACEUTICAL INDUSTRIES LIMITED, Mumbai, Maharashtra (IN)

(72) Inventors: Varinder KUMAR, Mohali (IN); Shavej AHMAD, Lucknow (IN); Romi Barat SINGH, Varanasi (IN); Kaushal NAYYAR, Gurgaon (IN); Mohan PRASAD, Gurgaon (IN)

(21) Appl. No.: 15/114,497

(22) PCT Filed: Jan. 26, 2015
(86) PCT No.: PCT/IB2015/050587

§ 371 Date: Jul. 27, 2016

(30) Foreign Application Priority Data
Jan. 28, 2014 (IN) 252/DEL/2014

(51) Int. Cl. A61K 9/00; A61K 9/20; A61K 31/197

(52) U.S. Cl. CPC A61K 9/0065; A61K 31/197; A61K 9/2013; A61K 9/2095; A61K 9/2027; A61K 9/2031; A61K 9/2054; A61K 9/2059; A61K 9/2009

(57) ABSTRACT

The present invention relates to stabilized gastroretentive tablets comprising pregabalin, one or more swellable polymers, a pH modifier, and other pharmaceutically acceptable excipients. It also relates to processes for the preparation of said stabilized gastroretentive tablets of pregabalin.

### FIELD OF THE INVENTION

The present invention relates to stabilized gastroretentive tablets comprising pregabalin, one or more swellable polymers, a pH modifier, and other pharmaceutically acceptable excipients. It also relates to processes for the preparation of said stabilized gastroretentive tablets of pregabalin.

### BACKGROUND OF THE INVENTION

Pregabalin, as disclosed in U.S. Pat. No. 6,197,819, is chemically designated as (S)-3-(aminomethyl)-5-methylhexanoic acid.

Pregabalin is not uniformly absorbed throughout the gastrointestinal tract, and is predominantly absorbed from the stomach and the upper part of the intestine. In such instances, it is beneficial to develop gastroretentive tablets that are retained in the upper parts of the gastrointestinal tract for prolonged periods of time.

Several attempts have been made in the prior art to provide gastroretentive dosage forms of pregabalin. U.S. Publication No. 2007/0269511 discloses a pharmaceutical composition comprising pregabalin, a matrix forming agent comprising polyvinyl acetate and polyvinylpyrrolidone, and a swelling agent comprising cross-linked polyvinylpyrrolidone, wherein the pharmaceutical composition is adapted for once-daily dosing.

PCT Publication No. WO 2010/143052 discloses a gastroretentive floating tablet of pregabalin comprising one or more water insoluble components, wherein the water insoluble component is preferably a combination of ethyl cellulose and hydrogenated castor oil.

However, one major problem with pregabalin formulations is the tendency to form an undesired cyclic lactam during manufacture and/or shelf life. Several attempts have been made in the prior art to reduce this tendency of pregabalin to form the corresponding lactam and provide stable formulations thereof.

U.S. Pat. No. 7,309,719 discloses a stabilized pharmaceutical composition consisting of gabapentin or pregabalin and a neutral a-amino acid as a stabilizer.

U.S. Publication No. 2009/0156677 discloses the use of a humectant as a stabilizer in pharmaceutical compositions containing pregabalin.

In view of the aforesaid, it is necessary to provide stabilized gastroretentive tablets of pregabalin that are substantially free of the lactam impurity. The present inventors have surprisingly found that the addition of a suitable pH modifier to gastroretentive tablets of pregabalin substantially reduces the formation of the undesired lactam impurity, thereby resulting in improved stability.

### SUMMARY OF THE INVENTION

The present invention relates to stabilized gastroretentive tablets comprising pregabalin that are substantially free of the lactam impurity. The stabilized gastroretentive tablets comprise pregabalin, one or more swellable polymers, a pH modifier, and other pharmaceutically acceptable excipients.

### DETAILED DESCRIPTION OF THE INVENTION

A first aspect of the present invention provides a stabilized gastroretentive tablet comprising pregabalin, one or more swellable polymers, a pH modifier, and other pharmaceutically acceptable excipients.

According to one embodiment of the above aspect, the swellable polymers are selected from the group comprising cellulosic polymers, polyalkylene oxides, polysaccharides, acrylic acid polymers, vinyl pyrrolidone polymers, and combinations thereof.

According to another embodiment of the above aspect, the pH modifier is selected from the group comprising magnesium oxide, sodium acetate, trisodium citrate, meglumine, trisodium orthophosphate, sodium bicarbonate, sodium hydroxide, and combinations thereof.

According to another embodiment of the above aspect, the pharmaceutically acceptable excipients are selected from the group comprising diluents, binders, disintegrants, lubricants/glidants, and combinations thereof.

According to another embodiment of the above aspect, the tablet is substantially free of the lactam impurity.

According to another embodiment of the above aspect, the tablets are prepared by the processes of direct compression, dry granulation, or wet granulation.

The term “pregabalin,” as used herein, includes pregabalin and salts, polymorphs, hydrates, solvates, prodrugs, chelates, and complexes thereof.

The term “gastroretentive tablet,” as used herein, refers to a tablet which is capable of staying in the stomach for a prolonged period of time, and therefore is capable of releasing pregabalin in the stomach for a time period longer than when delivered as a conventional tablet.

The term “stabilized,” as used herein, implies that the tablet is substantially free of the lactam impurity.

The term “lactam,” as used herein, refers to the undesired degradation product produced by intramolecular condensation reaction of the γ-amino group and the carboxylic acid group of pregabalin. This cyclic lactam of pregabalin is chemically 4-isobutyl-pyrrolidin-2-one.

The term “substantially free of lactam,” as used herein, implies that the lactam content does not exceed 0.6% by weight of lactam, preferably 0.4% by weight of lactam, more preferably 0.2% by weight of pregabalin.

The term “swellable polymers,” as used herein, refers to polymers that swell in the presence of gastric fluids. This swelling increases the size of the tablet to such an extent so as to provide retention of the tablet in the stomach of a patient. The swellable polymers that may be used in the present invention are selected from the group comprising cellulosic polymers, polyalkylene oxides, polysaccharides, acrylic acid polymers, vinyl pyrrolidone polymer, and combinations thereof Cellulosic polymers include methyl cellulose, hydroxymethyl cellulose, hydroxyethyl cellulose, hydroxypropyl cellulose, hydroxypropyl methyl cellulose, ethyl cellulose, sodium carboxymethyl cellulose, cross-linked sodium carboxymethyl cellulose, calcium carboxymethyl cellulose, and combinations thereof. Polyalkylene oxides include polyethylene oxide, such as that available under the trade name Polyox®. Polysaccharides include starch and starch-based polymers, chitosan, agar, alginates, carrageenan, furcellaran, guar gum, gum arabic, gum tragacanth, karaya gum, locust bean gum, pectin, dextran, gellan gum, rhamsan gum, welan gum, xanthan gum, propylene glycol alginate, hydroxypropyl guar, and combinations thereof. Vinyl pyrrolidone polymers include cross-linked polyvinylpyrrolidone and crospovidone.

Suitable pH modifiers are selected from the group comprising magnesium oxide, sodium acetate, trisodium citrate, meglumine, trisodium orthophosphate, sodium bicarbonate, sodium hydroxide, and combinations thereof.

The tablets of the present invention comprise other pharmaceutically acceptable excipients that are routinely used and are selected from the group comprising diluents, binders, disintegrants, lubricants/glidants, and combinations thereof.

Suitable diluents are selected from the group comprising microcrystalline cellulose; silicified microcrystalline cellulose; lactose; glucose; natural, modified, or pregelatinized starch; mannitol; sorbitol; and combinations thereof.

Suitable binders are selected from the group comprising povidone, methyl cellulose, ethyl cellulose, low-substituted hydroxypropyl cellulose, hydroxypropyl methyl cellulose, acacia, guar gum, alginic acid, dextrin, maltodextrin, polyvinyl alcohol, gelatin, starch, and combinations thereof.

Suitable disintegrants are selected from the group comprising sodium carboxymethyl cellulose; low-substituted hydroxypropyl cellulose; carboxymethyl cellulose; calcium carboxymethyl cellulose; cross-linked polyvinyl pyrrolidone; microcrystalline cellulose; natural, modified, or pregelatinized starch; gums; and combinations thereof.

Suitable lubricants/glidants are selected from the group comprising colloidal silicon dioxide, talc, stearic acid, magnesium stearate, zinc stearate, calcium stearate, sodium stearyl fumarate, hydrogenated castor oil, and combinations thereof.

The tablets described herein may be prepared by conventional processes using commonly available equipment. The process may comprise direct compression, wet granulation, or dry granulation.

The tablets of the present invention may be further coated with one or more non-functional coatings. The coating may comprise one or more film-forming polymers and coating additives.

Examples of film-forming polymers include ethyl cellulose, hydroxypropyl methyl cellulose, hydroxypropyl cellulose, methylcellulose, carboxymethyl cellulose, hydroxymethyl cellulose, hydroxyethyl cellulose, cellulose acetate, hydroxypropyl methyl cellulose phthalate, cellulose acetate phthalate, cellulose acetate trimellitate, waxes, and methacrylic acid polymers such as Eudragit®. Alternatively, commercially available coating compositions comprising film-forming polymers marketed under various trade names, such as Opadry®, may also be used.

Coating additives may be selected from the group comprising binders, plasticizers, opacifiers, coloring agents, and lubricants.

Examples of plasticizers include acetylated triacetin, triethyl citrate, tributyl citrate, glycerol tributyrate, diacetylated monoglyceride, polyethylene glycols, propylene glycol, sesame oil, acetyl tributyl citrate, acetyl triethyl citrate, diethyl oxalate, diethyl phthalate, diethyl maleate, diethyl fumarate, dibutyl succinate, diethyl malonate, dioctyl phthalate, dibutyl sebacate, and combinations thereof.

Examples of opacifiers include titanium dioxide, talc, calcium carbonate, behenic acid, cetyl alcohol, and combinations thereof

Coloring agents include any FDA approved color for oral use.

Specific examples of solvents for granulation or coating include water, acetone, ethanol, methanol, isopropyl alcohol, methylene chloride, and combinations thereof.

Coating may be performed by applying the coating composition as a solution, suspension, or blend using any conventional coating technique known in the art such as spray coating in a conventional coating pan or fluidized bed processor, dip coating, or compression coating

The tablets may be dispensed in packs made with usual packaging materials like high-density polyethylene (HDPE) bottles or blister packs. The package may additionally contain a desiccant.

The invention may be further illustrated by the following examples, which are for illustrative purposes only and should not be construed as limiting the scope of the invention in any way.

### EXAMPLES 1-5

| | Quantity (% w/w) | | | | |
|---|---|---|---|---|---|
| Ingredients | Example 1 | Example 2 | Example 3 | Example 4 | Example 5 |
| Pregabalin | 26.72 | 33.00 | 33.00 | 33.00 | 33.00 |
| Crospovidone | 25.02 | 25.00 | 31.00 | 30.88 | 30.75 |
| Hydroxypropyl | — | 25.00 | 29.00 | 28.87 | 28.75 |
| methyl cellulose | | | | | |
| Kollidon ® SR | 22.83 | — | — | — | — |
| Polyethylene oxide | 20.00 | — | — | — | — |
| Maltodextrin | — | 10.00 | — | — | — |
| Acrylic acid | 4.94 | 6.00 | 6.00 | 6.00 | 6.00 |
| polymer | | | | | |
| Magnesium oxide | — | — | — | 0.25 | 0.50 |
| Sodium acetate | — | — | — | — | — |
| Trisodium citrate | — | — | — | — | — |
| Magnesium stearate | 0.49 | 1.00 | 1.00 | 1.00 | 1.00 |

EXAMPLES 6-11

| | Quantity (% w/w) | | | | | |
|---|---|---|---|---|---|---|
| | Example | Example | Example | Example | Example | Example |
| Ingredients | 6 | 7 | 8 | 9 | 10 | 11 |
| Pregabalin | 33.00 | 33.00 | 33.00 | 33.00 | 33.00 | 33.00 |
| Crospovidone | 30.63 | 30.50 | 29.50 | 28.50 | 30.50 | 30.50 |
| Hydroxypropyl | 28.62 | 28.50 | 27.50 | 26.50 | 28.50 | 28.50 |
| methyl cellulose | | | | | | |
| Kollidon ® SR | — | — | — | — | — | — |
| Polyethylene | — | — | — | — | — | — |
| oxide | | | | | | |
| Maltodextrin | — | — | — | — | — | — |
| Acrylic acid | 6.00 | 6.00 | 6.00 | 6.00 | 6.00 | 6.00 |
| polymer | | | | | | |
| Magnesium | 0.75 | 1.00 | 3.00 | 5.00 | — | — |
| oxide | | | | | | |
| Sodium | — | — | — | — | 1.00 | — |
| acetate | | | | | | |
| Trisodium | — | — | — | — | — | 1.00 |
| citrate | | | | | | |
| Magnesium | 1.00 | 1.00 | 1.00 | 1.00 | 1.00 | 1.00 |
| stearate | | | | | | |

### Procedure

1. Each ingredient was sifted through mesh #20.

2. All the ingredients of step 1, except magnesium stearate, were blended together for 15 minutes.

3. The mixture of step 2 was blended with magnesium stearate for 5 minutes.

Blends prepared as per the above procedure were kept for 21 days at 40° C./75% RH and tested for lactam formation. The resultant stability data is provided in Table 1.

TABLE 1
Stability Data of Blends Prepared as per Examples 1-11

Percentage of Lactam

| Example | Initial | 21 days (40° C./75% RH) |
|---|---|---|
| 1 | 0.0040 | 0.240 |
| 2 | 0.0036 | 0.280 |
| 3 | 0.0100 | 0.260 |
| 4 | ND* | 0.080 |
| 5 | ND* | 0.060 |
| 6 | ND* | 0.030 |
| 7 | 0.0026 | 0.014 |
| 8 | 0.0010 | 0.016 |
| 9 | 0.0100 | 0.050 |
| 10 | ND* | 0.190 |
| 11 | ND* | 0.190 |

(*Not detectable)

The formulas of Examples 1, 2 and 3 do not include a pH modifier, and therefore serve as reference examples. The stability data demonstrates the addition of a pH modifier reduces the lactam formation.

### EXAMPLES 12-15

| | Quantity (% w/w) | | | |
|---|---|---|---|---|
| | Example | Example | Example | Example |
| Ingredients | 12 | 13 | 14 | 15 |
| Pregabalin | 32.04 | 32.04 | 32.04 | 33.00 |
| Crospovidone | 30.10 | 29.13 | 30.10 | 29.00 |
| Hydroxypropyl methyl | 30.58 | 28.15 | 29.12 | 29.00 |
| cellulose | | | | |
| Acrylic acid polymer | 3.88 | 3.88 | 4.76 | 4.00 |
| Meglumine | — | 3.40 | — | — |
| Trisodium | — | — | 0.58 | 4.50 |
| orthophosphate | | | | |
| Magnesium stearate | 0.49 | 0.49 | 0.49 | 0.50 |
| Opadry ® pink | 2.91 | 2.91 | 2.91 | — |
| Purified water | q.s. | q.s. | q.s. | — |

### Procedure for Examples 12, 13, and 14

1. All the ingredients, except magnesium stearate, were sifted through sieve #20 and blended for 15 minutes.

2. Magnesium stearate was sifted through sieve #25.

3. The blend of step 1 was blended with the material of step 2 for 5 minutes.

4. The blend of step 3 was compressed into a tablet using appropriate tooling.

5. Opadry® pink was dispersed in purified water and stirred for 45 minutes.

6. The tablets of step 4 were coated with the dispersion of step 5 in a perforated coating pan.

### Procedure for Example 15

1. Trisodium orthophosphate was dissolved in purified water.

2. The solution of step 1 was sprinkled on hydroxypropyl methyl cellulose to uniformly adsorb on it.

3. The material of step 2 was dried in a tray dryer at 40° C.

4. The remaining ingredients, except magnesium stearate, were sifted through sieve #20 and blended with material of step 3 for 15 minutes.

5. Magnesium stearate was sifted through sieve # 25.

6. The blend of step 4 was blended with the material of step 5 for 5 minutes.

7. The blend of step 6 was compressed into a tablet using appropriate tooling.

The tablets thus obtained were kept in HDPE bottles at 40° C./75% RH for 6 months and tested for lactam formation. The resultant stability data is provided in Table 2.

TABLE 2
Stability Data of Tablets Prepared as per Examples 12-15

| | Percentage of Lactam | | | | |
|---|---|---|---|---|---|
| Examples | Initial | 1 month | 2 months | 3 months | 6 months |
| 12 | 0.01 | 0.11 | 0.20 | 0.31 | 0.55 |
| 13 | 0.01 | 0.10 | 0.19 | 0.28 | 0.41 |
| 14 | 0.01 | 0.10 | 0.18 | 0.27 | 0.44 |
| 15 | — | 0.11 | 0.19 | 0.19 | 0.36 |

Example 12 is a reference example that does not contain a pH modifier. From the above data, it is evident that the tablets containing a pH modifier have reduced levels of lactam as compared to tablets without a pH modifier.

We claim:

1. A stabilized gastroretentive tablet comprising pregabalin, one or more swellable polymers, a pH modifier, and other pharmaceutically acceptable excipients.

2. The stabilized gastroretentive tablet according to claim 1, wherein the swellable polymers are selected from the group comprising cellulosic polymers, polyalkylene oxides, polysaccharides, acrylic acid polymer, vinyl pyrrolidone polymer, and combinations thereof.

3. The stabilized gastroretentive tablet according to claim 1, wherein the pH modifier is selected from the group comprising magnesium oxide, sodium acetate, trisodium citrate, meglumine, trisodium orthophosphate, sodium bicarbonate, sodium hydroxide, and combinations thereof.

4. The stabilized gastroretentive tablet according to claim 1, wherein the other pharmaceutically acceptable excipients are selected from the group comprising diluents, binders, disintegrants, lubricants/glidants, and combinations thereof.

5. The stabilized gastroretentive tablet according to claim 1, wherein the tablet is substantially free of the lactam impurity.

6. The stabilized gastroretentive tablet according to claim 1, wherein the tablet is prepared by direct compression, dry granulation, or wet granulation.
